(19) United States
(12) Patent Application Publication
(10) Pub. No.: US 2026/0210964 A1
(43) Pub. Date: Jul. 23, 2026

(54) METHOD FOR DETECTING DIVIDING CANCER CELLS USING PHOSPHO-Ki67 (Ser 2344) AS MITOTIC BIOMARKER

(71) Applicant: KOC UNIVERSITESI, Istanbul (TR)

(72) Inventors: Nazan SANER, Istanbul (TR); Nurhan OZLU SICAKKAN, Istanbul (TR); Ceyda Seren CEYHAN, Istanbul (TR)

(73) Assignee: KOC UNIVERSITESI, Istanbul (TR)

(21) Appl. No.: 19/132,836

(22) PCT Filed: Nov. 25, 2022
(86) PCT No.: PCT/TR2022/051357

§ 371 Date: May 26, 2025

(51) Int. Cl. G01N 33/575; G01N 33/50; G01N 33/543; G01N 33/68

(52) U.S. Cl. CPC G01N 33/57595; G01N 33/5091; G01N 33/54366; G01N 33/6893; G01N 2440/14; G01N 2474/10; G01N 2474/20

(57) ABSTRACT

A use of a biomarker which is for detection of dividing cancer cells based on the mitosis specific phosphorylation of Ki67 at serine 2344 is provided. A reagent (an antibody) against the phosphorylation of Ki67 at serine 2344 and a kit including the reagent are also provided for determining the presence, absence or progression of cancer in a subject.

### CROSS REFERENCE TO THE RELATED APPLICATIONS

This application is the national phase entry of International Application No. PCT/TR2022/051357, filed on Nov. 25, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBAP286_Sequence_Listing.xml, created on 05/23/2025, and is 17,619 bytes in size.

### TECHNICAL FIELD

The present invention discloses and claims the use of a biomarker which is for detection of dividing cancer cells based on the mitosis specific phosphorylation of Ki67 at serine 2344.

### BACKGROUND

Uncontrolled cell proliferation is a hallmark of cancer [1]. Almost 40 years ago, mice were immunized with the nuclei isolated from the Hodgkin's lymphoma cell line, and in this study, a monoclonal antibody Ki67 was developed against a nuclear antigen that is present only in proliferating cells, but not in quiescent cells [2]. Once the protein was discovered, it was named after the antibody, Ki67. The origin of name comes from the city where the study was conducted (Kiel, Germany) and the number of clone in 96-well plate (#67) [3]. As Ki67 was found to be expressed throughout the cell cycle, except for G0, which is a cellular quiescence state [4], Ki67 became a valuable proliferation marker and prognostic tool in clinical management of various types of cancer such as breast, prostate, kidney, pancreas and brain [5-9].

In the Chinese patent document CN112661842, an anti-Ki67 specific monoclonal antibody is disclosed. The antibody has been reported to be suitable for immunohistochemical detection in tissue samples of breast cancer, lung cancer, lymphoma, cervical cancer, and other malignant tumors. Although Ki67 is present at all stages of the cell cycle (G1, S, G2, and mitosis), Ki67 levels vary between different cell cycle stages [10, 11]. Ki67 was shown to be degraded during G0 and G1, whereas generated from S phase until mitosis. In addition, the single-cell analysis revealed that the level of Ki67 is not only dependent on the cell cycle stage, but also the time consumed at G0 before entering the cell cycle phase [11]. Therefore, even in a proliferating state, cells may possess a large spectrum of Ki67 levels due to their present cell cycle stage and history. This may lead to a variation in signals obtained from immunohistochemical staining of tissues with anti-Ki67 antibodies and so, brings difficulties in assessing cells as Ki67 positive or negative in tumor tissues. For this reason, novel biomarkers that can detect the proliferating cells only at cell division is necessary to eliminate the false results due to the variation in Ki67 levels during cell cycle.

It is very important to predict the progression of tumor and the survival of patient in oncology. Tumor type, stage and mitotic activity are the major criteria to estimate the prognosis of disease [12]. Mitotic activity, which is a parameter to assess the rate of tumor growth, is usually determined in hematoxylin and eosin (H&E) stained, formalin fixed, paraffin embedded tissue sections. However, the mitotic count using H&E stained slides can be very time-consuming and prone to errors due to the morphological imitation of pyknotic or apoptotic cells to mitotic cells [12-14]. For this reason, researchers started testing alternative mitotic biomarkers in tissue to avoid the technical difficulties related to H&E staining.

Phosphorylation is a reversible post-translational modification that can modulate a protein's structure and function, degradation rate, subcellular localization and binding affinities to other proteins [15]. Lately, immunohistochemical staining of phospho-Histone H3 (PHH3) gained a significant interest as a mitotic marker in tissue sections and its prognostic power in cancer is tested [13, 16-19]. Aurora B, which is one of the master regulators of cell division, phosphorylates Histone H3 not only at serine 10, but also at serine 28 at mitosis [20]. For this reason, PHH3 antibodies are widely used as mitotic biomarkers in research and are produced by leading companies.

Ki67 is also one of the proteins that are regulated in time and space in a cell cycle dependent manner. It translocates from nucleoli to the external surface of condensed chromosome upon entry to the mitosis [21]. An early study reported that this regulation is associated with the mitosis-specific phosphorylation of Ki67 [22]. Ki67 has the highest score as a protein regulated by phosphorylation. It is phosphorylated on more than 100 mitotically upregulated sites [23, 24].

Ki67 has structurally an atypical repeat region, which is composed of moderately conserved 16 tandem repeats of around 110 amino acids. Based on the conservation scores of repeats and mass spectrometry data of Ki67 phosphorylation sites, phospho-Ki67 (Thr 1943) antibody was developed by a research group in Japan [25]. The phosphorylation of threonine residue at 1943 on Ki67 was shown to appear at prophase, to reach the highest level at metaphase and to fade out through anaphase. The phosphorylation was not observed at interphase. Mitosis specific staining of phospho-Ki67 (Thr 1943) antibody was lost upon depletion of Ki67 in HeLa cells. In addition to phospho-Ki67 (Thr 1943) antibody, a phospho-Ki67 (Thr 1335) antibody was generated by a biotech company, Affinity Biosciences. A closer look into the alignment of amino acid sequences of repeats reveal that Thr 1335 (repeat #3) and Thr 1943 (repeat #8) share similar motifs on different repeats (FIG. 1). Therefore, there is a high possibility that the two very similar phosphopeptides were used to develop these antibodies, which recognize the conserved threonine phosphorylation sites on different repeats. However, the potential mitosis specific phosphorylation of threonine residue at 1335 on Ki67 was not experimentally tested. The specificity of phospho-Ki67 (Thr 1335) antibody was also not assessed in Ki67 depleted cells.

Despite advances in biomarkers, there is still a scarcity of mitotic biomarkers that have a great potential to bring solutions to the technical issues and to improve the diagnosis and prognosis of cancer. These needs and other needs are satisfied by the present invention.

### SUMMARY

Here, an antibody was developed against the cell division specific phosphorylation of Ki67 at serine 2344. Ki67 was found to be phosphorylated at serine 2344 from prophase until telophase, which is tightly specific to cell division. The aim of this invention is to repurpose Ki67 as a mitotic marker apart from being a proliferation marker in cancer histopathology.

The present invention relates to a biomarker and a method for using the biomarker to detect highly dividing cancer cells based on the cell division specific phosphorylation of Ki67 at serine 2344 in the repeat domain.

According to the present invention, a reagent (an antibody) has been developed against the phosphorylation of Ki67 at serine 2344 and the detection of highly dividing cancer cells with this reagent has been discovered.

The present invention relates to a cancer diagnosis method to be used for prognosis and diagnosis of cancer in a subject with the antibody developed to recognize phosphorylation of Ki67 at serine 2344. The phospho-Ki67 (Ser 2344) antibody specifically recognizes the phosphorylation of serine residue at 2344 and potentially the relevant serine residues that share similar flanking motifs at other repeats of Ki67 (FIG. 1). The phosphorylation of Ki67 at serine 2344 persists longer than the phosphorylation of histone H3 (Ser 10) during cell division (FIG. 5). This potentially makes phospho-Ki67 (Ser 2344) a better and sensitive biomarker than PHH3 in detecting dividing cancer cell in the tissue. The number of dividing cells giving signal will be higher in the case of phospho-Ki67 (Ser 2344). Therefore, this biomarker, phospho-Ki67 (Ser 2344) is a promising molecular target for the diagnosis and prognosis of cancer.

As cancer cells divide abnormally in tissue, there is a high potential of the developed antibody to be successful in cancer cell detection. The possibility of an improved cancer diagnosis and/or prognosis by using phospho-Ki67 (Serine 2344) brings an innovation to this field.

By means of the method of use the discovered biomarker and the developed antibody together, the detection of cancer cells from prophase until telophase has provided innovation and ease of use in the detection of dividing cancer cells.

The present invention relates to a new method that can be used to detect dividing cancer cells, and also aims to provide effective monitoring of the chemotherapeutic effect of anti-mitotic drugs, which are frequently used in cancer treatment.

This invention will allow pathologists to perform more accurate and precise tumor grading and cancer patients to receive more personalized treatment. The immunohistochemical staining of tumor tissue sections with phospho-Ki67 (Ser 2344) antibody will allow the calculation of the mitotic index, which is a significant parameter in assessing the progression of cancer and estimating the survival of patient.

Further aspect of the present invention relates to a nontherapeutic use of the biomarker (antibody) for the monitoring, isolating or screening (imaging) of cells in the mitosis phase, and also proliferative disorders such as cancer.

Another aspect of the present invention is directed to a kit for determining the presence, absence or progression of cancer in a subject including a reagent (herein antibody) that selectively binds to at least one cancer biomarker (herein phospho-Ki67 (Ser 2344)). According to the present invention said kit includes a reagent (an antibody) that specifically recognizes the phosphorylation at serine 2344 on Ki67 and further includes chemicals for the detection of antibody binding, a positive and negative control sample.

In addition to being a potentially strong biomarker in clinical studies, the phospho-Ki67 (Ser 2344) antibody can also serve as a cell division marker in basic science to distinguish this specific cell cycle stage in cell biology research field.

This object and other objects of this invention become apparent from the detailed discussion of the invention that follows.

### BRIEF DESCRIPTION OF THE DRAWINGS

The present invention is illustrated in the accompanying figures wherein;

FIG. 1 is the sequence alignment of highly conserved 16 tandem repeats on Ki67. Arrows point the cell division specific phosphorylation sites (Ser 1131, Ser 1253, Ser 1376, Ser 1861, Ser 2105, Ser 2344, Ser 2708, Ser 2828) that were identified in our phosphoproteomic study [23]. The peptide sequence that was used for the development of our phospho-Ki67 (Ser 2344) antibody and its counterparts on the other repeats are framed. The sequences flanking the phosphorylation sites of Ki67 at threonine 1335 and threonine 1943 show a high degree of similarity. Multiple sequence alignment was achieved using one of the EMBL-EBI search and sequence analysis tools [26], Clustal Omega (1.2.4) [27] and visualized in Jalview 2 [28]. The sequences numbered as repeat #1 to repeat #16 in FIG. 1 are as shown in SEQ ID NOS: 1-16.

FIG. 2 is an illustration of the specific staining of HeLa Kyoto cells with the invented phospho-Ki67 (Ser 2344) antibody during cell division. HeLa Kyoto and HeLa Kyoto-Ki67 KO (knockout) cells were fixed and stained with the invented phospho-Ki67 (Ser 2344), Ki67 and tubulin antibodies. DNA was stained with Hoechst. Scale Bar, 10 μm.

FIG. 3 is an illustration of immunofluorescence staining of non-cancer RPE1 cells with the invented phospho-Ki67 (Ser 2344) antibody during cell division. Cells were fixed and stained with the invented phospho-Ki67 (Ser 2344), Ki67 and tubulin antibodies. DNA was stained with Hoechst. Scale Bar, 10 μm.

FIG. 4 is an illustration of the Western blot analysis of both Ki67 and the invented phospho-Ki67 (Ser 2344) antibodies in a cell cycle dependent manner. Wild type and Ki67 KO HeLa Kyoto cells were synchronized at interphase (I) and mitosis (M). Tubulin was used as a loading control and phospho-Histone H3 (Ser 10) was employed as a mitosis marker.

FIG. 5 is an illustration of the temporal comparison of Ki67 phosphorylation at serine 2344 and histone H3 phosphorylation at serine 10 during cell division in HeLa Kyoto cells. Cells were fixed and stained with the phospho-Ki67 (Ser 2344), phospho-Histone H3 (Ser 10) and tubulin antibodies. DNA was stained with Hoechst. Scale Bar, 10 μm.

FIG. 6 is an illustration of Ki67 phosphorylation at threonine 1335 during cell division in HeLa Kyoto cells. Cells were fixed and stained with the phospho-Ki67 (Thr 1335), phospho-Histone H3 (Ser 10) and tubulin antibodies. DNA was stained with Hoechst. Scale Bar, 10 μm.

### DETAILED DESCRIPTION OF THE EMBODIMENTS

The present invention is the discovery of a biomarker for detecting dividing cancer cells and an antibody, which has a potential of detecting dividing cancer cells by means of the aforementioned biomarker. With this antibody, monitoring of dividing cancer cells will be possible, which will improve diagnosis and prognosis of cancer.

The present invention is related to a biomarker of mitosis (cell division) specific phosphorylation of Ki67 at serine 2344 and a reagent to detect dividing cancer cells.

The present invention is a method for detecting dividing cells in a biological sample in the presence of a mitotic biomarker and includes the following steps:
providing a biological sample and preparation of the biological extract from the sample,
contacting the biological extract with an antibody that specifically binds to a post-translationally modified biomarker protein in mitosis, wherein the antibody specifically recognizes the phosphorylation on Ki67 protein at serine 2344,
detecting the presence of the biomarker in the biological sample by qualitative or quantitative methods,
thereby detecting mitotic cancer and normal cells during cell division.

As used herein, the term “biological sample” or “sample” refers to any of the cancerous or healthy (non-cancerous) cells, cell sections, tissues or organs. The sample may be obtained from a subject by routine procedures known to the person skilled in the art (e.g., biopsy). The sample may contain a single or different type of cells at different cell cycle stages cell section, tissue or organ, and the sample can be taken from any part of a subject (patient). The sample may be fresh or frozen or pre-processed (eg formalin-fixed paraffin-embedded tissues). According to the present invention, the method, detecting the presence of the biomarker in the biological sample is performed by analyzing a biological sample from a subject, wherein the subject is a human.

As used herein, the term “subject” or “patient” refers to any mammal. As used here, the definition of “a dividing cancer cell” more specifically emphasizes the dividing cancer cell in the mitosis stage (M).

In the present invention, in the step of providing a biological sample and preparing the biological extract from the sample, to isolate or obtain cells or cell section, the sample is lysed. The isolated cells are lysed in a lysis solution, the proteins are isolated from the lysis solution, the isolated proteins are preferably incubated in a digestion solution containing trypsin to break down the proteins into peptides, and the peptides are purified from the resulting mixture.

In the preferred embodiment of the present invention, the cancer types that are detected, predicted or monitored by using the mitotic biomarker of the invention are cancer types wherein those cancer cells containing phosphorylated Ki67 (Ser 2344) protein. Samples can be taken from the liver, biliary tract, colon, ovary, prostate, lung, mesothelioma, chest, stomach, kidney, pancreas, uterus or cervix regions.

Determination of the presence or the level of the phosphorylated Ki67 (Ser 2344) protein by immunological methods using an antibody specifically recognizing the phosphorylation on Ki67 protein at serine 2344 is preferred.

Immunoassays may be qualitative (positive or negative) or quantitative (amount measured). According to the present invention, an antibody which specifically recognizes the phosphorylation at serine 2344 on Ki67 protein can be used in immunoassays, such as Western blots, enzyme-linked immunosorbent assay (ELISA), radioimmunoassay, immunohistochemical assay, immunoprecipitation, or other immunochemical assays known in the art. These assays can measure or determine the presence of complex formation between the mitotic biomarker and the antibody which specifically binds to the biomarker.

In the present invention, in the step of determining the presence of the biomarker by qualitative or quantitative methods, the detection of the presence of the biomarker by antibody is performed by any of the above-mentioned antigen-antibody detection methods.

An increase in the amount of the biomarker in said sample or extract is determined by comparison with a control sample. Furthermore, the present invention relates to a method of evaluating whether a subject is afflicted with cancer, the method includes the following steps:
determining the level of the biomarker in a sample obtained from said subject; and
comparing the level of the biomarker with the level of the biomarker in a control sample, wherein a difference between the level of biomarkers is an indication that the subject is afflicted with cancer.

In another embodiment, the present invention relates to a method of diagnosing cancer in a subject includes the following steps:
isolating the biological extract from a biological sample obtained from a subject,
measuring the level of phospho-Ki67 (Ser 2344),
comparing the presence or the level of phospho-Ki67 (Ser 2344) in the biological sample with the level of the biomarker in a control sample, and
diagnosis cancer if the level of phospho-Ki67 (Ser 2344) in the biological sample is increased relative to the control biological sample.

Preferably, the isolating biological extract from a biological sample step includes contacting the biological extract with an antibody (a reagent) wherein the antibody specifically recognizes the phosphorylation at serine 2344 on Ki67.

The present invention also relates to methods and kits for the qualitative and/or quantitative detection of the presence of a biomarker in a sample to identify a subject with cancer, and in particular to detect cells in mitosis.

These examples are intended to be representative of specific embodiments of the invention and are not intended as limiting the scope of the invention.

### SPECIFIC EMBODIMENTS

### Example 1 Discovery of Phospho-Ki67 (Ser 2344)

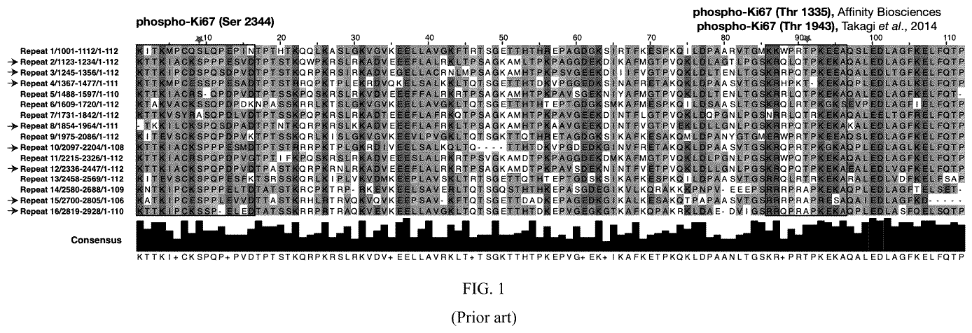

A quantitative phosphoproteomic study conducted in our laboratory identified eight phosphorylation sites (Ser 1131, Ser 1253, Ser 1376, Ser 1861, Ser 2105, Ser 2344, Ser 2708, Ser 2828) during cell division [23], which share a conserved motif in the repeat domain of Ki67 (FIG. 1). Based on the amino acid conservation scores of sequences that include the serine phosphorylation sites, the peptide sequence for the antibody development was determined as KTTKIACK(pS)PQPDPVD (SEQ ID NO: 17), where serine at 2344 is a phosphorylated serine (phospho-serine) (FIG. 1).

### Example 2 Development of Antibody Against Phospho-Ki67 (Ser 2344)

For the development of phospho-Ki67 (Ser 2344) antibody, we utilized custom antibody production service from Davids Biotechnologie GMbH (Germany). First, a phosphorylated peptide was synthesized based on the consensus sequence (KTTKIACK(pS)PQPDPVD, SEQ ID NO: 17), which was later used to immunize animals and produce antibodies. The non-phosphorylated form of the same peptide was also synthesized for the purification step. After the peptide synthesis, the phosphorylated peptide was conjugated to a carrier and used for immunization of two rabbits. The collected serum from rabbits was subjected to affinity chromatography using synthetic phospho peptides to get rid of unspecific antibodies. Then, the fraction was depleted from peptide specific antibodies using the non-phosphorylated peptides and only phospho-specific antibodies were obtained. The concentration of affinity purified and depleted phospho-Ki67 (Ser 2344) antibody is 0.2 mg/ml.

### Example 3 Detection of the Phospho-Ki67 (S2344) Throughout the Cell Cycle

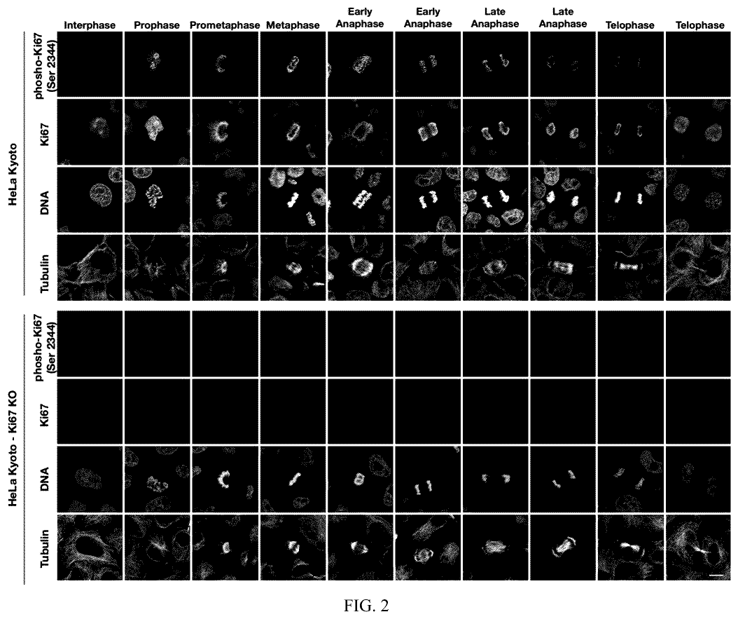

The cell division specific phosphorylation of serine at 2344 on Ki67 has been shown by a quantitative phosphoproteomic study [23]. Therefore, the immunofluorescence staining of HeLa Kyoto cells with our phospho-Ki67 (Ser 2344) antibody was expected to be specific to dividing cells, but not to the interphase cells. Indeed, the phosphorylation first appears in prophase cells when chromosomes condensate, then it gradually decreases during chromosome segregation at anaphase and disappears as the daughter chromosomes decondense (FIG. 2).

It should be noted that Ki67 is present throughout the whole cell cycle, but the modified form of Ki67 (phospho Ki67 at Ser 2344) was only observed in mitotic cells. When we stained Ki67 knockout (Ki67 KO) cells, in which Ki67 gene was disrupted and Ki67 is no longer present [29], phospho-Ki67 (Ser 2344) signal was not detected in any mitotic cells (FIG. 2). These results highlight the specificity of our phospho-Ki67 (Ser 2344) antibody.

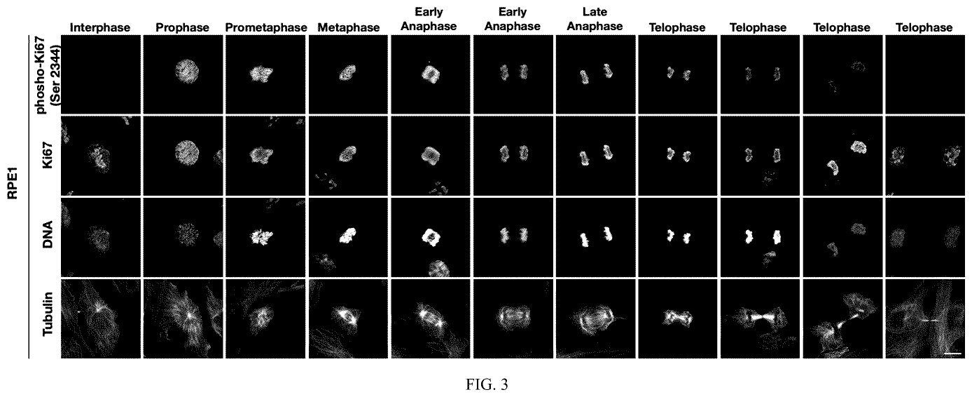

In parallel to cancer cells, we tested the specificity of our phospho-Ki67 (Ser 2344) antibody in non-cancer human retinal pigment epithelial cells (RPE1 cell line) which were immortalized with hTERT expression. Although Ki67 could be observed at all stages of cell cycle, the phosphorylation was again specific to cell division and decreased from anaphase to telophase (FIG. 3).

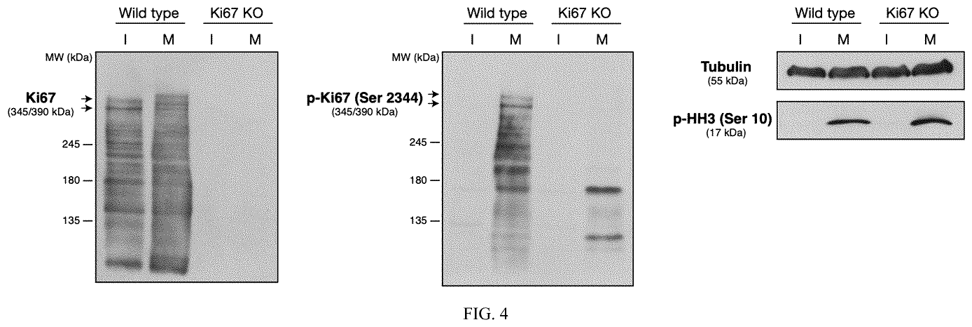

Next, we attempted to further validate the specificity of our phospho-Ki67 (Ser 2344) antibody and assess its application in Western blot analysis. For this, we first synchronized both wild type and Ki67 KO HeLa Kyoto cells at interphase and mitosis. The Western blotting results confirmed the absence of Ki67 in Ki67 KO cells and its presence both at interphase and mitosis in wild type cells. The heavy phosphorylation of Ki67 during mitosis results in a band shift. Our phospho-Ki67 (Ser 2344) antibody specifically detects the phosphorylated Ki67 only in mitotic wild type cells, but not in Ki67 KO cells (FIG. 4). These data support the results of immunofluorescence staining with our phospho-Ki67 (Ser 2344) antibody and reveal the successful application of phospho-Ki67 (Ser 2344) antibody in immunoblotting.

### Example 4 the Comparison of Temporal Regulation of Phospho-Histone H3 (Ser 10) and Phospho-Ki67 (Ser 2344)

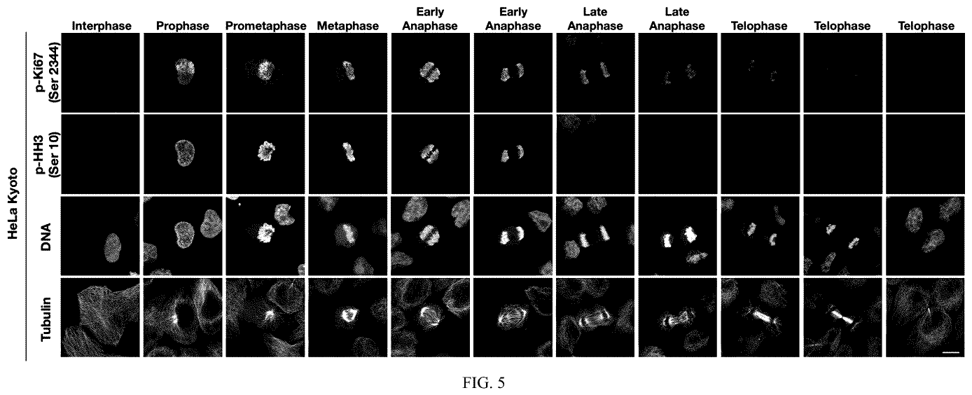

After confirming the cell division specificity of our phospho-Ki67 (Ser 2344) antibody, we compared its temporal regulation with a well-known mitosis marker, PHH3. It is very striking that the phosphorylation of Ki67 at serine 2344 persists longer than the phosphorylation of Histone H3 at serine 10, which sharply disappears at late anaphase (FIG. 5). This suggests that our phospho-Ki67 (Ser 2344) antibody has a higher potential to detect more dividing cancer cells in tissue when compared to PHH3, as the phosphorylation of Ki67 at serine 2344 gradually decreases after mitosis exit.

### Example 5 the Cell Cycle-Dependent Regulation of Phospho-Ki67 (Thr 1335) in Respect to Phospho-Histone H3 (Ser 10)

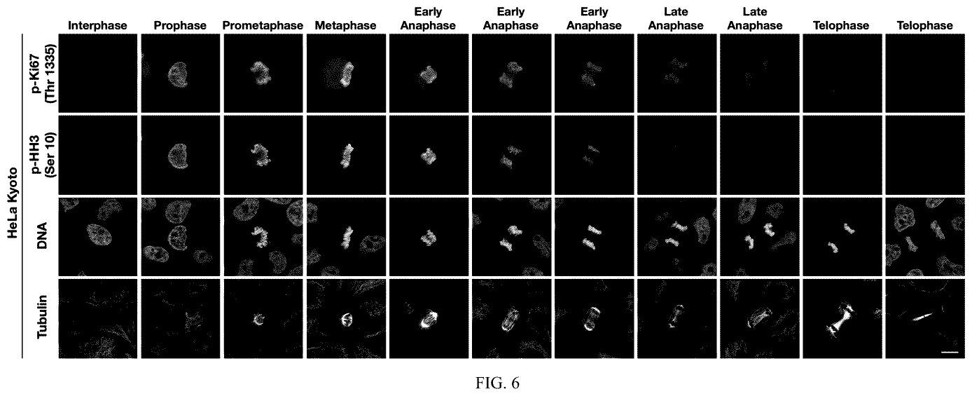

We then tested whether Ki67 phosphorylation at threonine 1335 is cell division specific or not, with respect to Histone H3 phosphorylation at serine 10. Although the phosphorylation of Ki67 at threonine 1335 keeps longer on the chromosomes than the phosphorylation of Histone H3 at serine 10, it is dramatically reduced at late anaphase and disappears in telophase cells (FIG. 6). The comparison of phosphorylation patterns at threonine 1335 and serine 2344 (FIG. 5 and FIG. 6) reveals that the phospho-Ki67 (Ser 2344) antibody recognizes a temporally distinct phosphorylation, which is maintained at higher levels in late anaphase until telophase. As the host of both phospho-Ki67 (Ser 2344) and phospho-Ki67 (Thr 1335) antibodies are rabbit, it was not possible to directly compare the temporal regulations of two phosphorylation sites (Ser 2344 and Thr 1335) on Ki67 with immunofluorescence staining.

### Example 6 Materials and Methods

### Cell Culture and Synchronizations

HeLa Kyoto cells were maintained in DMEM high glucose supplemented with 10% fetal bovine serum (FBS) and 1% penicillin-streptomycin (P/S). hTERT-RPE1 cells were grown in DMEM/F-12 1:1 mixture supplemented with 10% FBS, 2 mM L-glutamine and 1% P/S. All cells were cultured at 37° C. in a humidified atmosphere containing 5% CO₂. Cells were synchronized at interphase with a single thymidine block. Briefly, cells were treated with 2 mM thymidine for 20 h at 37° C. For mitotic arrest, cells were released from thymidine block for 4 h and incubated in complete medium containing 10 μM S-trityl-L-cysteine (STLC) (164739; Sigma-Aldrich) for 16 h.

### Cell Lysis and Western Blotting

For Western blotting, the cells were lysed in RIPA buffer (pH:7.5) containing 50 mM Tris, 150 mM NaCl, 0.1% SDS, 0.5% sodium deoxycholate, 1% Triton X-100, complete EDTA-free protease inhibitor cocktail (Roche), and PhosSTOP phosphatase inhibitor mixture (Roche). Protein samples were prepared in 5× Laemmli sample buffer containing 100 mM DTT. 5% and 10% SDS-PAGE gels were used for the separation of proteins. The wet transfer system (Bio-Rad) was used to transfer proteins to nitrocellulose membranes. After blocking with 5% BSA in 0.1% Tween 20 containing 1×TBS overnight at 4° C., the membranes were incubated with primary antibodies overnight at 4° C. and with secondary antibodies at RT for 1 h. The membranes were rinsed with 0.1% Tween 20 containing 1×TBS buffer for 5 min three times after antibody incubations. Proteins were visualized with the ECL Western Blotting Substrate system (32106; Pierce).

The primary antibodies used for immunoblotting were as the following. 1:200 rabbit anti-phospho-Ki67 (Ser 2344), 1:2,000 rabbit anti-Ki67 (ab16667, Abcam), 1:2,000 mouse anti-tubulin (3873S, Cell Signaling), 1:300 rabbit anti-phospho-histone H3 (Ser 10) (sc-8656-R; Santa Cruz Biotechnology). 1:2,000 HRP conjugated anti-mouse (7076, Cell Signaling) and anti-rabbit (7074, Cell Signaling) IgGs were used as secondary antibodies.

### Immunofluorescence Staining and Microscopy

For immunostaining, cells seeded on glass coverslips were fixed in 4% PFA in 1×PBS for 15 min. Coverslips were washed three times with PBS-0.1% Triton X-100 buffer for 5 min. After blocking with 2% BSA in PBS-0.1% Triton X-100, coverslips were incubated with primary antibodies at RT for 3 h and with secondary antibodies at RT for 1 h. The primary antibodies used for immunofluorescence staining were as the following. 1:500 rabbit anti-phospho-Ki67 (Ser 2344), 1:1,000 rabbit anti-phospho-Ki67 (Thr 1335) (AF3665, Affinity Biosciences), 1:1,000 mouse anti-Ki67 (9449, Cell Signaling), 1:500 rat anti-tubulin (ab6160, Abcam), 1:1000 mouse anti-phospho-histone H3 (Ser 10) (9706; Cell Signaling), 1:1,000 Alexa Fluor 488-, 568- and 633 conjugated anti-rabbit, anti-rat and anti-mouse IgGs (Life Technologies), respectively were used as secondary antibodies. Hoechst (33258, Sigma-Aldrich) was used to visualize DNA.

The coverslips were washed three times with PBS-0.1% Triton X-100 buffer for 5 min after antibody incubations. Coverslips were embedded in Mowiol mounting medium (81381, Sigma-Aldrich).

The images of fixed samples were acquired in a format of 1024×1024 pixels in XY settings with Leica DMi8/SP8 (LAS X Software) laser scanning confocal microscope using the 63× Plan Apo 1.4 NA oil-immersion objective. Single focal planes were used in the figures. Images were not deconvoluted.

### REFERENCES

[1]. Hanahan, D. and R. A. Weinberg. 2011. ‘Hallmarks of Cancer: The Next Generation’, Cell. 144, 646-674.
[2]. Gerdes, J., U. Schwab, H. Lemke, and H. Stein. 1983. ‘Production of a mouse monoclonal antibody reactive with a human nuclear antigen associated with cell proliferation’, International Journal of Cancer. 31, 13-20.
[3]. Scholzen, T. and J. Gerdes. 2000. ‘The Ki-67 protein: from the known and the unknown.’, Journal of Cellular Physiology. 182, 311-322.
[4]. Gerdes, J., H. Lemke, H. Baisch, H. H. Wacker, U. Schwab, and H. Stein. 1984. ‘Cell cycle analysis of a cell proliferation-associated human nuclear antigen defined by the monoclonal antibody Ki-67.’, The Journal of Immunology. 133, 1710-1715.
[5]. Pezzilli, R, S. Partelli, R. Cannizzaro, N. Pagano, S. Crippa, M. Pagnanelli, and M. Falconi. 2016. ‘Ki-67 prognostic and therapeutic decision driven marker for pancreatic neuroendocrine neoplasms (PNENs): A systematic review’, Advances in Medical Sciences 61, 147-153.
[6]. Menon, S. S., C. Guruvayoorappan, K. M. Sakthivel, and R. R. Rasmi. 2019. ‘Ki-67 protein as a tumour proliferation marker’, Clinica Chimica Acta. 491, 39-45.
[7]. Dowsett, M., T. O. Nielsen, R. A&apos; Hern, J. Bartlett, R. C. Coombes, J. Cuzick, M. Ellis, N. L. Henry, J. C. Hugh, T. Lively, L. McShane, S. Paik, F. Penault-Llorca, L. Prudkin, M. Regan, J. Salter, C. Sotiriou, I. E. Smith, G. Viale, J. A. Zujewski, and D. F. Hayes. 2011. ‘Assessment of Ki67 in Breast Cancer: Recommendations from the International Ki67 in Breast Cancer Working Group’, JNCI Journal of the National Cancer Institute. 103, 1656-1664.
[8]. Chen, W.-J., D.-S. He, R.-X. Tang, F.-H. Ren, and G. Chen. 2015. ‘Ki-67 is a Valuable Prognostic Factor in Gliomas: Evidence from a Systematic Review and Meta-analysis’, Asian Pacific Journal of Cancer Prevention. 16, 411-420.
[9]. Berlin, A., J. F. Castro-Mesta, L. Rodriguez-Romo, D. Hernandez-Barajas, J. F. González-Guerrero, I. A. Rodriguez-Fernández, G. González-Conchas, A. Verdines-Perez, and F. E. Vera-Badillo. 2017. ‘Prognostic role of Ki-67 score in localized prostate cancer: A systematic review and meta-analysis’, Urologic Oncology: Seminars and Original Investigations. 35, 499-506.
[10]. Sobecki, M., K. Mrouj, J. Colinge, F. Gerbe, P. Jay, L. Krasinska, V. Dulic, and D. Fisher. 2017. ‘Cell-Cycle Regulation Accounts for Variability in Ki-67 Expression Levels.’, Cancer Research. 77, 2722-2734.
[11]. Miller, I., M. Min, C. Yang, C. Tian, S. Gookin, D. Carter, and S. L. Spencer. 2018. ‘Ki67 is a Graded Rather than a Binary Marker of Proliferation versus Quiescence’, Cell Reports. 24, 1105-1112.
[12]. Klöppel, G. and S. La Rosa. 2018. ‘Ki67 labeling index: assessment and prognostic role in gastroenteropancreatic neuroendocrine neoplasms’, Virchows Archiv. 472, 341-349.
[13]. Puripat, N. and K. Loharamtaweethong. 2019. ‘Phosphohistone H3 (PHH3) as a surrogate of mitotic figure count for grading in meningiomas: a comparison of PHH3 (S10) versus PHH3 (S28) antibodies’, Virchows Archiv. 474, 87-96.
[14]. Huang, W., C. Nebiolo, K. Esbona, R. Hu, and R. Lloyd. 2020. ‘Ki67 index and mitotic count: Correlation and variables affecting the accuracy of the quantification in endocrine/neuroendocrine tumors’, Annals of Diagnostic Pathology. 48, 151586.
[15]. Cohen, P. 2000. ‘The regulation of protein function by multisite phosphorylation—a 25 year update.’, Trends in Biochemical Sciences. 25, 596-601.
[16]. Nielsen, P. S., R. Riber-Hansen, T. O. Jensen, H. Schmidt, and T. Steiniche. 2013. ‘Proliferation indices of phosphohistone H3 and Ki67: strong prognostic markers in a consecutive cohort with stage I/II melanoma’, Modern Pathology 26, 404-413.
[17]. Kim, J.-Y., H. S. Jeong, T. Chung, M. Kim, J. H. Lee, W. H. Jung, and J. S. Koo. 2017. ‘The value of phosphohistone H3 as a proliferation marker for evaluating invasive breast cancers: A comparative study with Ki67.’, Oncotarget. 8, 65064-65076.
[18]. Gerring, Z., J. F. Pearson, H. R. Morrin, B. A. Robinson, G. C. Harris, and L. C. Walker. 2015. ‘Phosphohistone H3 outperforms Ki67 as a marker of outcome for breast cancer patients.’, Histopathology. 67. 538-547.
[19]. Bosch, D. E., M. R. Kilgore, R. A. Schmidt, P. E. Swanson, M. H. Rendi, and O. H. Chang. 2017. ‘Comparison of Proliferation Markers Ki67 and Phosphohistone-H3 (pHH3) in Breast Ductal Carcinoma In Situ.’. Applied Immunohistochemistry & Molecular Morphology: AIMM. 25, 543-547.
[20]. Goto, H., Y. Yasui, E. A. Nigg, and M. Inagaki. 2002. ‘Aurora-B phosphorylates Histone H3 at serine28 with regard to the mitotic chromosome condensation.’, Genes to Cells: Devoted to Molecular & Cellular Mechanisms. 7, 11-17.
[21]. Endl, E. and J. Gerdes. 2000. ‘The Ki-67 Protein: Fascinating Forms and an Unknown Function’, Experimental Cell Research. 257, 231-237.
[22]. Endl, E. and J. Gerdes. 2000. ‘Posttranslational modifications of the Ki-67 protein coincide with two major checkpoints during mitosis.’, Journal of Cellular Physiology. 182, 371-380.
[23]. Karayel, Ö., E. Sanal, S. H. Giese, Z. C. Uretmen Kagiali, A. N. Polat, C.-K. Hu, B. Y. Renard, N. Tuncbag, and N. Özlü 2018. ‘Comparative phosphoproteomic analysis reveals signaling networks regulating monopolar and bipolar cytokinesis.’, Scientific Reports. 8, 2269.
[24]. [24]. Dephoure, N., C. Zhou, J. Villen, S. A. Beausoleil, C. E. Bakalarski, S. J. Elledge, and S. P. Gygi. 2008. ‘A quantitative atlas of mitotic phosphorylation.’, Proceedings of the National Academy of Sciences of the United States of America. 105, 10762-10767.
[25]. Takagi, M., Y. Nishiyama, A. Taguchi, and N. Imamoto. 2014. ‘Ki67 antigen contributes to the timely accumulation of protein phosphatase 1γ on anaphase chromosomes.’, Journal of Biological Chemistry. 289, 22877-22887.
[26]. Madeira, F., M. Pearce, A. R. N. Tivey, P. Basutkar, J. Lee, O. Edbali, N. Madhusoodanan, A. Kolesnikov, and R. Lopez. 2022. ‘Search and sequence analysis tools services from EMBL-EBI in 2022’, Nucleic Acids Research. 50, W276-W279.
[27]. Sievers, F. and D. G. Higgins. 2018. ‘Clustal Omega for making accurate alignments of many protein sequences’, Protein Science. 27, 135-145.
[28]. Waterhouse, A. M., J. B. Procter, D. M. A. Martin, M. Clamp, and G. J. Barton. 2009. ‘Jalview Version 2—a multiple sequence alignment editor and analysis workbench’, Bioinformatics. 25, 1189-1191.
[29]. Cuylen, S., C. Blaukopf, A. Z. Politi, T. Müller-Reichert, B. Neumann, 1. Poser, J. Ellenberg, A. A. Hyman, and D. W. Gerlich. 2016. ‘Ki-67 acts as a biological surfactant to disperse mitotic chromosomes’, Nature. 535, 308-312.

What is claimed is:

1. A method for detecting dividing cells in a biological sample in a presence of a mitotic biomarker, comprising the following steps:
providing the biological sample and preparing a biological extract from the biological sample,
contacting the biological extract with an antibody that specifically binds to a post-translationally modified biomarker protein in mitosis, wherein the antibody specifically recognizes phosphorylation on a Ki67 protein at serine 2344,
detecting the presence of the mitotic biomarker in the biological sample by a qualitative or quantitative method,
thereby detecting a mitotic cancer and normal cells during cell division.

2. The method according to claim 1, wherein the qualitative or quantitative method is selected from the group consisting of Western blots, enzyme-linked immunosorbent assay (ELISA), radioimmunoassay, immunohistochemical assay, and immunoprecipitation assay.

3. The method according to claim 2, wherein the biological sample is selected from the group consisting of liver, biliary tract, colon, ovary, prostate, lung, mesothelioma, chest, stomach, kidney, pancreas, and uterus.

4. A kit for identifying a subject with cancer by detecting a presence of phosphorylated Ki67 (Ser 2344) in a sample, wherein the detection is performed by the method according to claim 1.

5. The kit according to claim 4, wherein the kit comprises the antibody.

6. The kit according to claim 5, wherein the kit further comprises chemicals for a detection of antibody binding.

7. The kit according to claim 6, wherein the kit further comprises a positive control sample and a negative control sample.

8. A reagent applied in the method according to claim 1 or comprised in a kit, wherein the kit is configured for identifying a subject with cancer by detecting a presence of phosphorylated Ki67 (Ser 2344) in a sample, the detection is performed by the method, and the reagent specifically recognizes the phosphorylation at serine 2344 on the Ki67 protein.

9. A method of diagnosing a cancer in a subject, comprising the following steps:
isolating a biological extract from a biological sample obtained from the subject,
measuring a level of phosphorylated Ki67 (Ser 2344),
comparing the level of phosphorylated Ki67 (Ser 2344) in the biological sample with a level of phosphorylated Ki67 (Ser 2344) in a control sample,
diagnosising the cancer if the level of phosphorylated Ki67 (Ser 2344) in the biological sample is increased relative to the control sample.

10. The method according to claim 9, wherein the step of isolating the biological extract from the biological sample comprises: contacting the biological extract with an antibody, wherein the antibody specifically recognizes phosphorylation at serine 2344 on Ki67.

11. The kit according to claim 4, wherein in the method, the qualitative or quantitative method is selected from the group consisting of Western blots, ELISA, radioimmunoassay, immunohistochemical assay, and immunoprecipitation assay.

12. The kit according to claim 11, wherein in the method, the biological sample is selected from the group consisting of liver, biliary tract, colon, ovary, prostate, lung, mesothelioma, chest, stomach, kidney, pancreas, and uterus.

13. The reagent according to claim 8, wherein in the method, the qualitative or quantitative method is selected from the group consisting of Western blots, ELISA, radioimmunoassay, immunohistochemical assay, and immunoprecipitation assay.

14. The reagent according to claim 13, wherein in the method, the biological sample is selected from the group consisting of liver, biliary tract, colon, ovary, prostate, lung, mesothelioma, chest, stomach, kidney, pancreas, and uterus.

15. The reagent according to claim 8, wherein the kit comprises the antibody.

16. The reagent according to claim 15, wherein the kit further comprises chemicals for a detection of antibody binding.

17. The reagent according to claim 16, wherein the kit further comprises a positive control sample and a negative control sample.

18. The kit according to claim 11, wherein the kit comprises the antibody.

19. The kit according to claim 12, wherein the kit comprises the antibody.

20. The kit according to claim 18, wherein the kit further comprises chemicals for a detection of antibody binding.


SEQUENCE LISTING (17 sequences)

SEQ ID NO: 1 - protein, 112 aa, synthetic construct
KITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTP

SEQ ID NO: 2 - protein, 112 aa, synthetic construct
KTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTP

SEQ ID NO: 3 - protein, 112 aa, synthetic construct
KTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTP

SEQ ID NO: 4 - protein, 111 aa, synthetic construct
KTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGLKELFQTP

SEQ ID NO: 5 - protein, 110 aa, Synthetic construct
KTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQT

SEQ ID NO: 6 - protein, 112 aa, Synthetic construct
KTAKVACKSSQPDPDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTP

SEQ ID NO: 7 - protein, 112 aa, synthetic construct
KTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTP

SEQ ID NO: 8 - protein, 111 aa, synthetic construct
TKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTP

SEQ ID NO: 9 - protein, 112 aa, synthetic construct
KITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTP

SEQ ID NO: 10 - protein, 108 aa, synthetic construct
KTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTP

SEQ ID NO: 11 - protein, 112 aa, synthetic construct
KTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTP

SEQ ID NO: 12 - protein, 112 aa, synthetic construct
KTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTP

SEQ ID NO: 13 - protein, 112 aa, synthetic construct
KITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAP

SEQ ID NO: 14 - protein, 109 aa, synthetic construct
KNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSET

SEQ ID NO: 15 - protein, 106 aa, synthetic construct
KATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKD

SEQ ID NO: 16 - protein, 110 aa, synthetic construct
KTTKIPCKSSPELEDTATSSKRRPRTRAQKVEVKEELLAVGKLTQTSGETTHTDKEPVGEGKGTKAFKQPAKRKLDAEDVIGSRRQPRAPKEKAQPLEDLASFQELSQTP

SEQ ID NO: 17 - protein, 16 aa, synthetic construct
  MOD_RES 9: note = phosphorylated serine
KTTKIACKSPQPDPVD